(19) United States
(12) Patent Application Publication
(10) Pub. No.: US 2026/0314946 A1
(43) Pub. Date: Oct. 15, 2026

(54) MICROCIRCULATORY BLOOD FLOW MONITORING SYSTEM AND MICROCIRCULATORY BLOOD FLOW MONITORING DEVICE

(71) Applicant: XUZHOU LIHUA ELECTRONIC TECHNOLOGY DEVELOPMENT CO., LIMITED, Xuzhou (CN)

(72) Inventors: Xinghuai FENG, Xuzhou (CN); Xiaoping ZHANG, Xuzhou (CN); Hui FENG, Ames, IA (US)

(21) Appl. No.: 19/338,857

(22) Filed: Sep. 24, 2025

(30) Foreign Application Priority Data
Nov. 10, 2024 (CN) 202411594859.X

(51) Int. Cl. A61B 5/026; G06T 7/00

(52) U.S. Cl. CPC A61B 5/0261; G06T 7/0012; A61B 2576/00; G06T 2207/10056; G06T 2207/30104

(57) ABSTRACT

A microcirculatory blood flow monitoring system includes a microcirculatory observation instrument, an acquisition module, a storage module, and a data processing module. The data processing module includes category data and a corresponding first-category indicator, and divides a microcirculatory blood flow velocity in a single vessel into different ranges based on a velocity magnitude, to form the first-category indicator corresponding to each velocity range, and the first-category indicator is corresponding to a velocity-related factor; and the velocity-related factor is assigned different values based on different blood flow velocity ranges; an assignment range for the velocity-related factor is between greater than or equal to 0 and less than or equal to 1, the velocity-related factor includes one or more of the following related sub-factors: a high-velocity blood flow-related sub-factor, a stagnant blood flow-related sub-factor, and a normal blood flow-related sub-factor.

### FIELD OF TECHNOLOGY

The present invention relates to a microcirculatory blood flow monitoring system, belonging to the technical field of microcirculatory blood flow monitoring.

### BACKGROUND

Currently, the International Consensus on Sepsis 3.0 points out that there are challenges in defining sepsis and septic shock. First, sepsis is a broad term used to describe a process that is not yet fully understood. So far, there have been no simple and clear clinical criteria or biological, imaging, or laboratory characteristics that can uniquely identify a patient with sepsis.

Because a pathogenic mechanism of sepsis is not yet fully understood, there are currently no “simple and clear” indicators for early sepsis infection in humans and mammals. Without early indicators of sepsis infection, early prediction of sepsis remains an international challenge. Similarly, there is no way to differentiate between early, middle, and late stages of sepsis, and no way to distinguish early general inflammation from sepsis (the International Consensus on Sepsis 3.0 notes: a working group attempted to distinguish sepsis from simple infection).

In fact, every disease has an early phase, and sepsis also has an early phase. Therefore, there is a significant clinical need for qualitative and quantitative indicators for ultra-early and early prediction of sepsis, as well as qualitative and quantitative indicators that can scientifically distinguish early general inflammation from sepsis and scientifically divide stages of sepsis.

Currently, infection is mainly determined clinically by blood, sputum, or urine cultures, as well as body temperature, X-ray, or CT scans. However, it takes a long time to perform blood, sputum, or urine cultures in clinical practice, and many cultures usually yield negative results, with a positive rate usually around 30% and not exceeding 50% at best. This leads to many infected patients not being diagnosed, resulting in significant uncertainty for clinical treatment.

Clinically, a method that can detect infection non-invasively, promptly, sensitively, and accurately is also expected to be provided, but currently, no such method or indicator exists.

Various major surgeries, such as cardiac surgery, a neurosurgery, thoracic surgery, and abdominal surgery, all carry a risk of postoperative infection, which usually results in a patient ultimately dying of infection even if after a successful surgery. Therefore, during a postoperative observation period, it is necessary to closely monitor possible wound infection, and the appearance of high-velocity blood flow in microcirculation indicates early infection at the wound, which needs immediate intervention.

Further, a common feature of different types of shocks is abnormal microcirculation, but there is currently no set of quantitative microcirculatory indicators to distinguish quantitative data characteristics of different types of shocks.

An ultimate goal in fluid resuscitation for critical illnesses is to restore microcirculatory perfusion. However, there is currently no comprehensive set of quantitative microcirculatory indicators and data to calculate and detect a state before and after restoration of normal microcirculatory perfusion.

In addition, after early resuscitation, it is necessary to first assess a patient's fluid (volume) responsiveness before determining whether to perform infusion. Therefore, assessment of fluid (volume) responsiveness during critical illness fluid resuscitation is extremely important. Commonly used methods and static and dynamic indicators for volume responsiveness assessment include passive leg raising (PLR), response to rapid fluid infusion, a blood pressure, a heart rate, central venous pressure (CVP), stroke volume (SV), stroke volume variation (SVV), pulse pressure variation (PPV), transthoracic echocardiography (TTE), transesophageal echocardiographic Doppler (TEE), observation of changes in aortic blood flow (ABF), ultrasonic cardiac output monitors (USCOM), or the like, which are all macrodynamic hemodynamic methods. Currently, there is no accurate quantitative indicator or method for microhemodynamics (microcirculation) volume responsiveness assessment in emergency and critical care.

Similarly, in critical illness and emergency rescue, and perioperative volume management, endpoints of fluid resuscitation currently rely on dynamic and static indicators of macrodynamic hemodynamics, such as central venous pressure (CVP), pulse pressure variation (PPV), stroke volume variation (SVV), and the like. There is also no quantitative indicator or method for microhemodynamics (microcirculation) that can more precisely determine a gold standard for individualized infusion quantity based on individual differences.

Additionally, antibiotics are chemical substances synthesized by some microorganisms that can inhibit or kill some pathogens. There is a plurality of types of antibiotics, and the efficacy of different antibiotics against the same pathogenic microorganism varies. Different pathogenic microorganisms also have different types of sensitivity to the same antibiotic, resulting in significant differences in clinical treatment outcomes. To more accurately target pathogenic microorganisms with medication and achieve optimal therapeutic effects, clinical practice usually employs bacterial culture combined with drug susceptibility testing to assess the sensitivity of the pathogenic microorganisms to various antibiotics. This allows selection of the most effective antibiotic for treating the pathogenic microorganisms in the patient, thereby achieving optimal therapeutic effects and shortening a course of disease of the patient.

Antibiotic sensitivity is usually determined by bacterial culture combined with the drug susceptibility testing. Antibiotic sensitivity testing can reveal the sensitivity of the pathogenic microorganism causing the disease to a particular antibiotic. Further, antibiotic resistance can also be tested, providing a basis for clinical medication guidance, which is of great clinical significance.

However, it takes an excessively long time to perform clinical blood, sputum, or urine cultures, making it difficult to obtain timely results to guide treatment. In addition, a plurality of cultures often yields negative results, with a positive rate usually around 30% and not exceeding 50% at best. This leads to a plurality of infected patients not being diagnosed, resulting in great uncertainty for clinical treatment. Clinically, it is also expected that quantitative indicators of microcirculatory blood flow after infection is used while the presence of bacteria in the blood is used, to promptly, sensitively, and accurately detect the sensitivity or resistance of a particular antibiotic to a specific bacterium, but currently such indicators do not exist.

For diseases prone to thrombosis due to hypercoagulable blood states, such as cerebral infarction, myocardial infarction, pulmonary embolism, lower limb venous thrombosis (thrombosis can also occur in sites such as the inferior vena cava, portal vein, intracranial veins, upper limb veins, and the like), it is also necessary to predict possible thrombosis timely using quantitative microcirculatory indicators, but currently such indicators do not exist.

### SUMMARY

To overcome the above shortcomings of the prior art, the present invention provides a microcirculatory blood flow monitoring system, and specifically provides a microcirculatory flow velocity quantitative indicator and a data detection and calculation system and method.

To avoid ambiguity, the present invention distinguishes several currently recognized types of shocks into three types based on different characteristics of a blood flow velocity: A first type is distributive (infectious) shock, where high cardiac output and low resistance manifest as high-velocity blood flow in microhemodynamics, resulting in “warm shock.” It is found that a cause of this “warm shock” is the Bernoulli effect, rather than the textbook explanation of warm shock based on a theory of collateral branch shunting. Because the Bernoulli principle of fluid dynamics was introduced into the analysis of a microcirculatory blood flow velocity for the first time by the inventor (Feng Xinghuai), so that, the inventor names this shock as “Feng Xinghuai-Bernoulli-Warm Shock” (see principle below).

A second type is a cold shock in distributive (infectious) shock, which is converted from the warm shock and is referred to as “infectious cold shock.” Both types are distributive shock or infectious shock.

A third type is a non-infectious, namely, a non-distributive shock, which includes a cardiogenic shock, an obstructive shock, a hypovolemic shock, a neurogenic shock, and an anaphylactic shock, collectively referred to as: the third type of shocks.

To avoid ambiguity, the blood flow velocity V in the present invention means a microcirculatory blood flow velocity V occurring in humans and mammals.

The present invention defines the microcirculatory high-velocity blood flow in the (internationally referred to as hyperdynamic blood flow: Hyperdynamic), which has the same meaning.

Further, the present invention defines the high-velocity blood flow as follows: Vd is a microcirculatory blood flow velocity exceeding a normal microcirculatory velocity limit V in humans under a resting state, namely, V greater than or equal to 1,000 μm/s, which is high-velocity blood flow or hyperdynamic blood flow Vd. The Hyperdynamic blood flow Vd is classified into three levels based on different ranges of the blood flow velocity: Vd1, Vd2, and Vd3, with specific connotations as specified below.
First level: the microcirculatory high-velocity blood flow velocity Vd1, where Vd1 is greater than or equal to 1,000 μm/s and less than 1,300 μm/s, which is high-velocity blood flow (hyperdynamic blood flow) Vd1 of suspected sepsis infection;
Second level: the microcirculatory high-velocity blood flow velocity Vd2, where Vd2 is greater than or equal to 1,300 μm/s and less than 1,500 μm/s, which is the high-velocity blood flow (hyperdynamic blood flow) Vd2 of highly suspected sepsis infection;
Third level: the microcirculatory high-velocity blood flow velocity Vd3, where Vd3 is greater than or equal to 1,500 μm/s, which can be clearly defined as microcirculatory high-velocity blood flow (hyperdynamic blood flow) of sepsis infection.

This high-velocity blood flow of sepsis infection exists only in true capillary exchange networks and microvenules with an oxygen exchange function. Blood flow occurring in any other vessels, regardless of velocity, is not included in the hyperdynamic blood flow or high-velocity blood flow in the present invention due to the lack of an oxygen molecule diffusion function resulting from thicker vessel walls, such as blood flow in anastomotic vessels and microarterioles in a blood microcirculatory network.

This high-velocity blood flow or hyperdynamic blood flow Vd occurring under the resting state tends to cause oxygen exchange disorders in thin-walled true capillaries and microvenules due to the Bernoulli effect of fluid dynamics, resulting in the “Feng Xinghuai-Bernoulli-Warm Shock.” If not treated, prolonged hypoxia will develop into an “infectious cold shock” and eventually multiple organ dysfunction syndrome (MODS). Therefore, careful observation, assessment, and calculation for this phenomenon and process are required.

To avoid ambiguity, the present invention defines sepsis infection and septic infection as belonging to the same category, but the septic infection can mean symptoms occurring in an early stage of sepsis.

Because there are currently no indicators for early sepsis, to facilitate understanding and acceptance by the medical community, the present invention defines early sepsis infection as septic infection.

The present invention uses the terms retention, stasis, stagnation, and congestion to uniformly express two phenomena in microcirculatory blood flow: cessation of flow and oscillation without forward movement. Oscillation without forward movement is also considered non-flowing.

The present invention uniformly defines a blood flow velocity V less than or equal to 90 μm/s as a “stagnant blood flow Vz.” Septic stagnant blood flow Vz defined in the present invention means blood flow transformed from sepsis high-velocity blood flow to stagnant blood flow, or blood flow stasis caused by other shock etiologies: Vz is microcirculatory blood flow with a velocity less than a lower limit V (less than or equal to 90 μm/s) of a normal microcirculatory blood flow velocity in humans.

Further, the stagnant blood flow Vz is classified into three levels based on different ranges of the blood flow velocity: Vz4, Vz5, and Vz6.
First level: the microcirculatory stagnant blood flow velocity Vz4. When Vz4 is less than or equal to 30 μm/s, Vz4 can be clearly defined as microcirculatory stagnant blood flow Vz4 of severe sepsis infection.
Second level: the microcirculatory stagnant blood flow velocity Vz5. When Vz5 is greater than 30 μm/s and less than or equal to 60 μm/s, Vz5 can be defined as microcirculatory stagnant blood flow Vz5 of highly suspected severe sepsis infection.
Third level: the microcirculatory stagnant blood flow velocity Vz6. When Vz6 is greater than 60 μm/s and less than or equal to 90 μm/s, Vz6 can be defined as microcirculatory stagnant blood flow Vz6 of suspected severe sepsis infection.

In the present invention, infection caused by septic stagnant blood flow Vz is defined as the “severe sepsis infection,” but Vz is also used for computational assessment of the severity of “other non-infectious shocks.”

In the present invention, a phenomenon in which all blood flow observed within the field of view of a microcirculatory instrument is microcirculatory stagnant blood flow velocity Vz4 is defined as “complete severe infection,” also referred to as “complete cold shock.” Vz4 is also applicable for computational assessment of “complete other non-infectious shocks.”

In the present invention, a phenomenon in which all blood flow observed within the field of view of a microcirculatory instrument is the stagnant blood flow velocity Vz5 is defined as “highly suspected complete severe infection,” also referred to as a “highly suspected complete cold shock.” Vz5 is also applicable for computational assessment of “other non-infectious types of highly suspected shocks.”

In the present invention, a phenomenon in which all blood flow observed within the field of view of a microcirculatory instrument is a microcirculatory stagnant blood flow velocity Vz6 is stipulated as “suspected complete severe infection,” also referred to as a “suspected complete cold shock.”

Vz6 is also applicable for computational assessment of “other suspected non-infectious shocks.”

The above complete severe infection Vz4 means an infectious shock as defined by International Sepsis 3.0.

In the present invention, the same set of indicators is used for “other non-infectious shocks,” namely Vz4, Vz5, and Vz6; and the symbol * denotes multiplication in the present invention.

In the present invention, a phenomenon in which all blood flow observed within the field of view of a microcirculatory instrument is high-velocity blood flow Vd1 (greater than or equal to 1,000 μm/s and less than 1,300 μm/s) is stipulated as “suspected complete moderate infection,” also referred to as a “suspected complete warm shock.”

In the present invention, a phenomenon in which all blood flow observed within the field of view of a microcirculatory instrument is high-velocity blood flow Vd2 (greater than or equal to 1,300 μm/s and less than 1,500 μm/s) is stipulated as “highly suspected complete moderate infection,” also referred to as a “highly suspected complete warm shock.”

In the present invention, a phenomenon in which all blood flow observed within the field of view of a microcirculatory instrument is high-velocity blood flow Vd3 (greater than or equal to 1,500 μm/s) is stipulated as “complete moderate infection”, also referred to as a “complete warm shock”.

In the present invention, a quantitative indicator for mild-to-moderate septic infection and a quantitative indicator for “Feng Xinghuai—Bernoulli—warm shock” caused by septic infection are defined using the same set of indicators.

In the present invention, a microcirculatory blood flow velocity V less than 1,000 μm/s and greater than 90 μm/s is defined as a “normal blood flow velocity Vc,” and a phenomenon in which all blood flow observed within the field of view of a microcirculatory instrument is a normal blood flow velocity Vc (less than 1,000 μm/s and greater than 90 μm/s) is stipulated as a “completely normal blood flow velocity.”

Refer to the international classification of capillary diameters and current international conventions. Microcirculatory vessels with a diameter less than or equal to 20 m are defined as small vessels, microcirculatory vessels with a diameter greater than 20 m and less than or equal to 60 m are defined as medium vessels, and microcirculatory vessels with a diameter greater than 60 m are defined as large vessels. Refer to the 2007 International Round Table Conference Minutes 2007-CC-How to evaluate the microcirculatory report of a round table conference, Critical Care Vol 11 No 5 De Backer et al.

The present invention defines, according to the theory of capillary autonomous movement and dilation-constriction in the field of microcirculatory, the division of microcirculatory blood flow into basal blood flow and pulse blood flow superimposed on the basal blood flow. The pulse blood flow is characterized by random rapid blood flow within vessels with stable basal blood flow, with features of randomness, a high frequency, and a flow velocity greater than an original vessel's basal blood flow velocity. The microcirculatory blood flow velocity mentioned in the present invention, especially microcirculatory high-velocity blood flow, is a basal flow with a stable average velocity, rather than randomly occurring pulse blood flow.

In the present invention, the analyzed microcirculatory images are stable, clear, and pressure artifact-free microcirculatory blood flow images continuously observed for at least 3-5 seconds.

In the present invention, commonly known methods are recommended for measuring the microcirculatory blood flow velocity, such as a space-time method, a red/white blood cell or plasma particle tracking method, namely, determining a velocity of particles by dividing a distance by time, and a light spot simulation tracking method.

For vessel diameter measurement, commonly known measurement methods are also used. To be specific, a software line-drawing method is used, where a straight line is drawn across a vessel diameter, and the length of the line is calculated as the vessel diameter by using software, or a section of a vessel's outer boundary contour is drawn, and an average diameter of this boundary contour is calculated as the vessel diameter by using a computer.

A method for selecting different blood flow velocity ranges is: selecting a recording M with the fastest blood flow velocity among all clear, stable, pressure-artifact-free microcirculation recordings acquired for the same measured object in the same session; selecting a vessel M1 with the fastest blood flow velocity from the recording M as a measured target, where a vessel diameter is not considered for this fastest blood flow velocity, which is not a velocity of pulse blood flow, but rather a basic blood flow velocity as described in the present invention; and measuring a velocity after selecting the vessel with the fastest blood flow velocity, then determining whether this fastest blood flow velocity falls within a range of high-velocity blood flow Vd1, Vd2, Vd3, stagnant blood flow Vz4, Vz5, Vz6, or normal blood flow velocity Vc, and determining a type of infection accordingly.

The present invention is implemented through the following technical solutions:
a microcirculatory blood flow monitoring system is provided, including a microcirculatory observation instrument, an acquisition module, a storage module, and a data processing module. The acquisition module observes blood flow and acquires blood flow data at one or more observable microcirculation sites on a subject using the microcirculatory observation instrument;
the storage module stores the blood flow data acquired by the acquisition module on a storage medium; the data processing module includes first category data and a corresponding first-category indicator A, and divides the first category data, namely, a microcirculatory blood flow velocity V in a single vessel, into different ranges based on a velocity magnitude, to form the first-category indicator A corresponding to each velocity range, and the first-category indicator A is corresponding to a velocity-related factor a; the velocity-related factor a is assigned different values based on different blood flow velocity ranges; an assignment range for the velocity-related factor a is between greater than or equal to 0 and less than or equal to 1, the velocity-related factor a includes one or more of the following related sub-factors: a high-velocity blood flow-related sub-factor a1, a stagnant blood flow-related sub-factor a2, and a normal blood flow-related sub-factor a3.

Further, the high-velocity blood flow sub-factor a1 is corresponding to high-velocity blood flow with the microcirculatory blood flow velocity V greater than 1,000 μm/s. The high-velocity blood flow is divided into three levels:
high-velocity Level 1 Vd3: a high blood flow velocity greater than or equal to 1,500 μm/s;
high-velocity Level 2 Vd2: a high blood flow velocity greater than or equal to 1,300 μm/s and less than 1,500 μm/s; and
high-velocity Level 3 Vd1: a high blood flow velocity greater than 1,000 μm/s and less than 1,300 μm/s.

The stagnant blood flow-related sub-factor a2 is corresponding to stagnant blood flow with the microcirculatory blood flow velocity V less than or equal to 90 μm/s, which is divided into three levels:
stagnant Level 1 Vz4: a stagnant blood flow velocity less than or equal to 30 μm/s;
stagnant Level 2 Vz5: a stagnant blood flow velocity greater than 30 μm/s and less than or equal to 60 μm/s; and
stagnant Level 3 Vz6: a stagnant blood flow velocity greater than 60 μm/s and less than or equal to 90 μm/s.

The normal blood flow-related sub-factor a3 is corresponding to normal blood flow with the microcirculatory blood flow velocity V less than or equal to 1,000 μm/s and greater than 90 μm/s.

According to the above different levels of velocities, corresponding sub-sub factors are assigned to the velocity-related sub-factors: the velocity-related sub-factor a1 is divided into sub-sub factors a1-1 corresponding to Vd1, a1-2 corresponding to Vd2, and a1-3 corresponding to Vd3; and the velocity-related sub-factor a2 is divided into sub-sub factors a2-1 corresponding to Vz4, a2-2 corresponding to Vz5, and a2-3 corresponding to Vz6.

Further, the data processing module further includes second category data and a corresponding second category indicator B; the second category indicator B is corresponding to a proportional factor b, an indicator of the proportional factor b means a ratio of an area occupied by a vessel in a single recording within a different flow velocity range to a single recording area within an instrument's field of view at a single location, or a ratio of a length of a vessel within a different flow velocity range to a total vessel length, or a ratio of a density of a vessel within a different flow velocity range to a density of all vessels within a single recording area, where the density means a value obtained dividing the total vessel length by the single recording area; the proportional factor b serves as both the second category data and the indicator; and a value range for the proportional factor b is between greater than or equal to 0 and less than or equal to 1.

Further, the data processing module further includes third category data and a corresponding third category indicator D; the third category data includes vessel diameter range classification, and the third category indicator D includes a diameter-related factor d corresponding to a vessel diameter range, where vessel data within a different diameter range is corresponding to a respective diameter-related factor d; the diameter-related factor d is assigned different values based on different vessel diameter ranges; and a value range for the diameter-related factor d is between greater than or equal to 0 and less than or equal to 1.

Further, the data processing module further includes a standard factor indicator g for high-velocity blood flow, stagnant blood flow, or normal-velocity blood flow, the standard factor indicator is g=1 when all blood flow velocities fall within a high-velocity blood flow range or all fall within a stagnant blood flow range; or the standard factor indicator is g=0 when all blood flow velocities fall within a normal-velocity blood flow range.

Further, the data processing module further includes fourth category data, namely, calculation data Qn % of related factors:
Qn % is calculated using any one of the following Formula 1-1, Formula 1-2, or Formula 1-3:

Qn %=(a*b) % Formula 1-1:

Qn %=(a*b*d) % Formula 1-2:

Qn %=(a*b*d*g) % Formula 1-3:
where n=1,2,3 . . . , which is a natural number sequence, when n=1, Q1% is a percentage of the high-velocity blood flow; when n=2, Q2% is a percentage of the stagnant blood flow; when n=3, Q3% is a percentage of the normal blood flow; a is the velocity-related factor, b is the proportional factor, d is the diameter-related factor, and g is the standard factor indicator.

Further, a value range of the flow-related velocity a is 0-1: as a blood flow velocity is approaching 1,500 μm/s from a lower value, a high-velocity blood flow-related sub-factor a1 is approaching 1; as the blood flow velocity is greater than 1,500 μm/s, a1 is assigned a value of 1; and as the blood flow velocity is approaching 0-30 μm/s from above 30 μm/s, the stagnant blood flow-related sub-factor a2 is approaching 1; within a 0-30 μm/s (comprising 30 μm/s) velocity range, a2=1, the normal blood flow-related sub-factor is a3=1;
a value range of the diameter-related factor d is 0-1: microcirculatory vessels with a diameter less than or equal to 20 m are classified as small vessels, microcirculatory vessels with a diameter greater than 20 m and less than or equal to 60 m are classified as medium vessels, and microcirculatory vessels with a diameter greater than 60 m are classified as large vessels. When high-velocity blood flow occurs in large, medium, and small vessels, a sub-factor related to d is d1=1, which is assigned within 0-1 in other cases. When stagnant blood flow occurs in the large, medium, and small vessels, the sub-factor related to d is d2=1, which is assigned within 0-1 in other cases.

Further, the data processing module further includes fifth-category data C, namely, multi-recording percentage average value data C; and C includes at least one sub-item C1; and
C1 is obtained by summing a single recording high-velocity blood flow percentage Q1 for all recordings of the same measured object in the same session and dividing a sum by the total number of recordings for the subject in the session, and C1 is referred to as a multi-recording high-velocity blood flow percentage average value C1, as shown in Formula 3,

$$C 1 = \{ [ {\sum }_{i = 1}^{t} Q 1 \% ( i ) ] / t \} * 100 \% ,$$

i=1,2,3 . . . t.; i represents a natural number sequence of recordings with Q1% present; for recordings without Q1%, a value is 0; and t represents the total number of recordings for the same measured object in the same session.

Further, the data processing module further includes fifth category data C, namely multi-recording percentage average value data C, and C includes at least two sub-items, C1 and C2;
C2: C2 is obtained by summing a stagnant blood flow percentage Q2% of each single recording stagnant blood flow indicator of each recording for the same measured object in the same session and by dividing a sum by the total number of all recordings of the same measured object in the same session, referred to as the multi-recording stagnant blood flow percentage average value C2, as shown in Formula 4,

$$C 2 = \{ [ {\sum }_{w = 1}^{t} Q 2 \% ( w ) ] / t \} * 100 \% ,$$

w=1,2,3 . . . t.; w represents a natural number sequence of recordings with Q2% present; for recordings without Q2%, a value is 0; and t represents the total number of recordings for the same measured object in the same session.

A microcirculatory blood flow monitoring device is provided, this device includes a microcirculatory observation instrument, a computer or another display device, software, a pressure control and image stabilization apparatus, an acquisition module, a storage module, and a data processing module. The acquisition module observes and records blood flow in one or more pressure artifact-free sites with clearly visible vessels at the sublingual mucosa or other observable microcirculation sites of a subject in a resting state, with the aid of software or other display methods, using the microcirculatory observation instrument and computer or another display device. The storage module stores recordings of one or more selected microcirculatory observation regions on a computer hard disk or other media using software or other means. The data processing module includes a first category indicator A, which divides a microcirculatory blood flow velocity V in a single vessel into different ranges based on a velocity magnitude, to form the first category indicator A corresponding to each velocity range, which is also referred to as a velocity-related factor a for ease of understanding and calculation; a is assigned different values based on different blood flow velocity ranges; and an assignment interval for a is between greater than or equal to 0 and less than or equal to 1.

Further, the data processing module includes: determining a value of a related proportional factor b of a second category indicator B for each single recording and a value of a diameter-related factor d thereof after a value of a for each recording is determined; an indicator of the proportional factor b means a ratio of an area occupied by a vessel in a single recording within a different flow velocity range to a single recording area within an instrument's field of view at a single location; or a ratio of a length of a vessel within a different flow velocity range to a total vessel length, or a ratio of a density of a vessel within a different flow velocity range to a density of all vessels within a single recording area, where the density means a value obtained by dividing a total length of a vessel by the single recording area; an assignment interval for b is between greater than or equal to 0 and less than or equal to 1; d is assigned different values based on different vessel diameter ranges, and an assignment interval for d is between greater than or equal to 0 and less than or equal to 1; and a value of Qn % is calculated using Formula 1; and then fifth category data C, C1 and C2, is calculated based on Formula 3 and Formula 4.

Further, the microcirculatory observation instrument is a microcirculatory microscope probe, the pressure control and image stabilization apparatus that is adjustable is installed on a microcirculatory microscope probe, the pressure control and image stabilization apparatus includes a positioning apparatus installed on a microcirculatory microscope elongated probe (1), the positioning apparatus includes a positioning apparatus front end (4) and a positioning apparatus rear end (6), both of the positioning apparatus front end (4) and the positioning apparatus rear end (6) are sleeves, centers of the positioning apparatus front end and the positioning apparatus rear end are each provided with a through hole, allowing the microcirculatory microscope elongated probe or a disposable tapered protective apparatus for the elongated probe to pass through, and the positioning apparatus rear end (6) is close to a microcirculatory instrument main unit (7); the positioning apparatus front end (4) and the positioning apparatus rear end (6) are fixed together or connected separately; the positioning apparatus is capable of reciprocating along an axial direction (2) of the elongated probe of the microcirculatory instrument main unit and is fixed after being adjusted to a suitable position, and the positioning apparatus front end (4) is provided with a flange (4-1) that serves as a bracket; the length of the pressure control and image stabilization apparatus on the microcirculatory microscope is adjusted so that the microcirculatory microscope elongated probe just contacts a sublingual mucosa and is blocked by outer lips, allowing clear images to be seen without excessive pressure causing artificial stagnant blood flow artifacts; and the pressure control and image stabilization apparatus is installed on the microcirculatory microscope probe to acquire stable, clear, and pressure-artifact-free microcirculation recordings from the subject at a resting state, which are stored on a computer hard disk or other media.

### Beneficial Effects of the Present Invention are as Follows

1. A distinct feature of microcirculatory blood flow in ultra-early and early stages of a septic shock is the occurrence of high-velocity blood flow. The microcirculatory quantitative indicators and the data detection and calculation system and method proposed in the present invention possess sufficient sensitivity, specificity, and accuracy of positive predictive values for the ultra-early and early prediction of septic infection as described above.

2. Various major surgeries, such as cardiac surgery, neurosurgery, thoracic surgery, and abdominal surgery, all face a potential issue of postoperative infection, which usually results in successful surgery but ultimately leads to patient death due to infection. Therefore, during a postoperative observation period, close monitoring of possible wound infection is required. The emergence of moderate or severe microcirculatory infection indicators suggests early or mid-to-late stage wound infection, requiring immediate rescue.

3. The severity of the septic infection and the severity of the shock are based on data C of a multi-recording percentage average value.

4. The microcirculatory quantitative indicators and the data detection and calculation system and method can assess the sensitivity or resistance of a bacterium to antibiotics.

5. In addition, an ultimate goal in acute and critical illness fluid resuscitation is to restore microcirculatory perfusion. Therefore, the microcirculatory perfusion also requires the microcirculatory flow velocity quantitative indicators and the data detection and calculation system and method to relatively accurately assess a final outcome of fluid resuscitation, which is the return of microcirculatory blood flow to a normal state.

6. In addition, the occurrence of microcirculatory high-velocity blood flow tends to cause tachycardia, which can easily lead to heart failure in patients with poor cardiac function. Therefore, a possibility of heart failure can be timely evaluated and preventive measures can be taken in advance by using the microcirculatory flow velocity quantitative indicators and the data detection and calculation system and method in the present invention.

7. Quantitative indicators and methods of microscopic hemodynamics (microcirculation) can more accurately determine a gold standard for individualized fluid infusion volume based on individual differences. During initial resuscitation, blood flow recovers from stasis to a high-velocity blood flow state. In a later stage of continued infusion, if blood flow stasis reappears, especially if pulse blood flow weakens or disappears, it indicates that an infusion volume has exceeded a body's tolerance limit. In addition, ECMO is used to rescue patients in the ICU, and a major reason for failure is DIC (disseminated intravascular coagulation). The DIC can be intuitively, promptly, accurately, and rapidly detected at the bedside by using the instrument in an early stage, which is a key auxiliary tool for ECMO success.

### BRIEF DESCRIPTION OF THE DRAWINGS

The present invention will be further described below with reference to drawings and embodiments.

FIG. 1 is a screenshot of a software interface of a microcirculatory monitoring system according to the present invention;

FIG. 2 is a schematic diagram of hardware of the microcirculatory monitoring system according to the present invention; and

FIG. 3 to FIG. 6 are schematic diagrams of a pressure control and image stabilization apparatus disposed in different manners on a microcirculatory microscope.

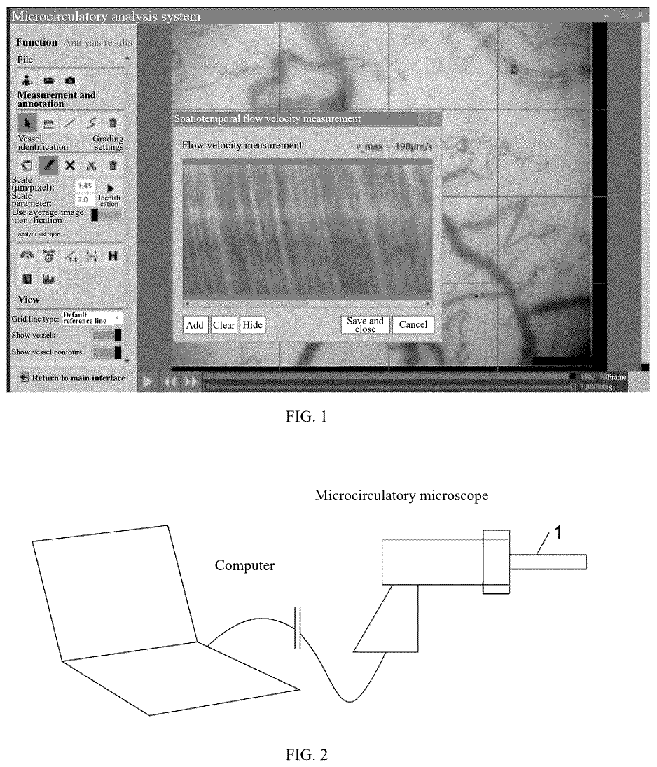

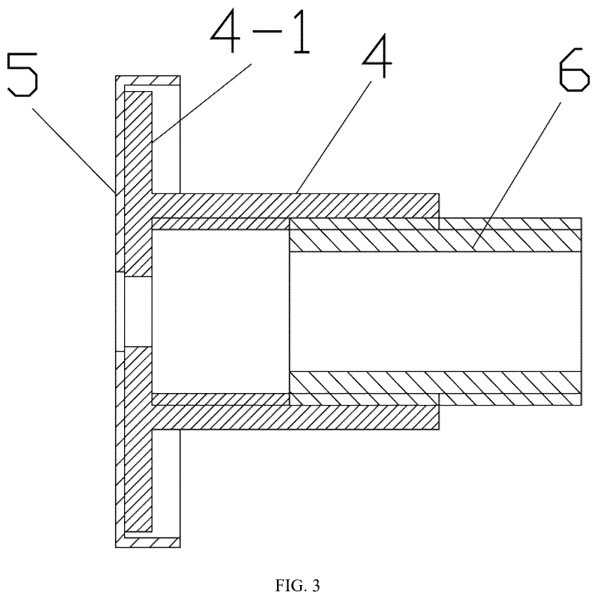

### DESCRIPTION OF THE EMBODIMENTS

Technical solutions in embodiments of the present invention are clearly and completely described below with reference to accompanying drawings in the specification. Apparently, the described embodiments are merely some rather than all of the embodiments of the present invention. The following description of at least one exemplary embodiment is merely illustrative and should not be construed as any limitation on the present invention and application or use of the present invention. Based on the embodiments of the present invention, all other embodiments obtained by those of ordinary skill in the art without creative effort fall within the protection scope of the present invention.

Technical solutions, methods, and devices known to those skilled in the art may not be discussed in detail, but should be regarded as part of the specification where appropriate.

In an embodiment, a microcirculatory flow velocity quantitative indicator and data detection and calculation system and method are provided, referred to as a microcirculatory blood flow monitoring system, including a computer or another display device, a microcirculatory observation instrument, software, an acquisition module, a storage module, and a data processing module. The acquisition module observes and records blood flow in one or more pressure artifact-free sites with clearly visible vessels at the sublingual mucosa or other observable microcirculation sites of a subject in a resting state, with the aid of software or other display methods, using the microcirculatory observation instrument and the computer or another display device.

The storage module stores recordings of one or more selected microcirculatory observation regions on a computer hard disk or other media using software or other means. The data processing module includes first category data and a corresponding first category indicator A, which divides the first category data, namely, a microcirculatory blood flow velocity V in a single vessel, into different ranges based on a velocity magnitude, to form the first category indicator A corresponding to each velocity range, which is corresponding to a velocity-related factor a for ease of understanding and calculation; a is assigned different values based on different blood flow velocity ranges; and an assignment interval for a is between greater than or equal to 0 and less than or equal to 1. The velocity-related factor a includes one or more of the following parameters: a high-velocity blood flow sub-factor a1, a stagnant blood flow sub-factor a2, and a normal blood flow sub-factor a3.

In an embodiment, the high-velocity blood flow sub-factor a1 is corresponding to high-velocity blood flow with the microcirculatory blood flow velocity V greater than 1,000 μm/s. A high-velocity blood flow indicator is divided into three levels:
high-velocity Level 1 Vd3: a high blood flow velocity greater than or equal to 1,500 μm/s;
high-velocity Level 2 Vd2: a high blood flow velocity greater than or equal to 1,300 μm/s and less than 1,500 μm/s; and
high-velocity Level 3 Vd1: a high blood flow velocity greater than 1,000 μm/s and less than 1,300 μm/s.

The stagnant blood flow-related sub-factor a2 is corresponding to stagnant blood flow with the microcirculatory blood flow velocity V less than or equal to 90 μm/s, which is divided into three levels:
stagnant Level 1 Vz4: a stagnant blood flow velocity less than or equal to 30 μm/s;
stagnant Level 2 Vz5: a stagnant blood flow velocity greater than 30 μm/s and less than or equal to 60 μm/s; and
stagnant Level 3 Vz6: a stagnant blood flow velocity greater than 60 μm/s and less than or equal to 90 μm/s.

The normal blood flow-related sub-factor a3 is corresponding to normal blood flow with the microcirculatory blood flow velocity V less than or equal to 1,000 μm/s and greater than 90 μm/s.

Velocity-related sub-factors a1, a2, and a3 are further assigned corresponding velocity-related sub-sub-factors based on the above velocities at different levels: a1 sub-sub-factors are a1-1, a1-2, a1-3; a2 sub-sub-factors are a2-1, a2-2, a2-3; and a3 sub-sub-factor is a3-1.

In an embodiment, the data processing module further includes second category data and a corresponding second category indicator B. The second category indicator B is corresponding to a relevant proportional factor b. An indicator of the proportional factor b means a ratio of an area occupied by a vessel in a single recording within a different flow velocity range to a single recording area within an instrument's field of view at a single location, or a ratio of a length of a vessel within a different flow velocity range to a total vessel length, or a ratio of a density of a vessel within a different flow velocity range to a density of all vessels within a single recording area, where the density means a value obtained by dividing the total vessel length by the single recording area. The proportional factor b serves as both the second category data and the indicator; and a value range for the proportional factor b is between greater than or equal to 0 and less than or equal to 1.

In an embodiment, the data processing module further includes third category data and a corresponding third category indicator D. The third category data includes vessel diameter range classification, and the third category indicator D includes a diameter-related factor d corresponding to a vessel diameter range, where vessel data within a different diameter range is corresponding to a respective diameter-related factor d; d is assigned different values based on different vessel diameter ranges; and a value range for the diameter-related factor d is between greater than or equal to 0 and less than or equal to 1.

In an embodiment, the data processing module further includes a standard factor g for high-velocity blood flow, stagnant blood flow, or normal-velocity blood flow. The standard factor indicator is g=1 when all blood flow velocities fall within a high-velocity blood flow range or all fall within a stagnant blood flow range; or the standard factor indicator is g=0 when all blood flow velocities fall within a normal-velocity blood flow range.

In an embodiment, the data processing module further includes fourth category data, namely calculation data Qn % from Formula 1: multiplying the flow velocity-related factor a, the proportional factor b, the diameter-related factor d, and the standard factor indicator g to obtain a percentage Qn % of high-velocity blood flow, stagnant blood flow, or normal blood flow for a single recording, as per Formula 1: Qn %=(a*b*d*g) %, n=1,2,3 . . . natural number sequence; Q1% is a percentage of high-velocity blood flow, Q1%=(a1*b*d*g) %, Q2% is a percentage of stagnant blood flow, Q2%=(a2*b*d*g) %, Q3% is a percentage of normal blood flow, and Q3%=(a3*b*d*g) %.

The data processing module further includes calculation data in Formula 2: single recording proportion data P, which includes Q1, Q2, and Q3; a relationship among Q1, Q2, and Q3 is proportional, as per Formula 2: P=Q1%:Q2%:Q3%. This proportional relationship displays a proportion of high-velocity blood flow, stagnant blood flow, and normal blood flow in a single recording.

In an embodiment, a value range of the flow-related velocity factor a in the data processing module is 0-1: a high-velocity blood flow-related sub-factor a1 is approaching 1 as a blood flow velocity is approaching 1,500 μm/s from a lower value,; and a1 is assigned a value of 1 as the blood flow velocity is greater than 1,500 μm/s; and as the blood flow velocity is approaching 0-30 μm/s from above 30 μm/s, the stagnant blood flow-related sub-factor a2 is approaching 1; within a 0-30 μm/s (including 30 μm/s) velocity range, a2=1, the normal blood flow-related sub-factor is a3=1.

A value range of the diameter-related factor d is 0-1: microcirculatory vessels with a diameter less than or equal to 20 m are classified as small vessels, microcirculatory vessels with a diameter greater than 20 m and less than or equal to 60 m are classified as medium vessels, and microcirculatory vessels with a diameter greater than 60 m are classified as large vessels. When high-velocity blood flow occurs in large, medium, and small vessels, a sub-factor related to d is d1=1, which is assigned within 0-1 in other cases. When stagnant blood flow occurs in large, medium, and small vessels, the sub-factor related to d is d2=1, which is assigned within 0-1 in other cases.

In an embodiment, the data processing module further includes fifth category data C, namely multi-recording percentage average value data C, and C contains at least one sub-item C1.
C1 is obtained by summing a single recording high-velocity blood flow percentage Q1 for all recordings of the same measured object in the same session and dividing a sum by the total number of recordings for the subject in the session, and C1 is referred to as a multi-recording high-velocity blood flow percentage average value C1, as shown in Formula 3,

$$C 1 = \{ [ {\sum }_{i = 1}^{t} Q 1 \% ( i ) ] / t \} * 100 \% ,$$

i=1,2,3 . . . t.; i represents a natural number sequence of recordings with Q1% present; for recordings without Q1%, a value is 0; and t represents the total number of recordings for the same measured object in the same session.

A physical meaning of Formula 3 is to aggregate a plurality of recordings into a single virtual recording using a percentage average value, with the percentage average value representing a high-velocity blood flow percentage of this virtual recording to indicate the severity across all recordings in this session.

The data processing module further includes fifth category data C, namely multi-recording percentage average value data C, and C includes at least two sub-items, C1 and C2.
C2: C2 is obtained by summing a stagnant blood flow percentage Q2% of each single recording stagnant blood flow indicator of each recording for the same measured object in the same session and by dividing a sum by the total number of all recordings of the same measured object in the same session, referred to as the multi-recording stagnant blood flow percentage average value C2, as shown in Formula 4,

$$C 2 = \{ [ {\sum }_{w = 1}^{t} Q 2 \% ( w ) ] / t \} * 100 \% ,$$

w=1,2,3 . . . t.; w represents a natural number sequence of recordings with Q2% present; for recordings without Q2%, a value is 0; and t represents the total number of recordings for the same measured object in the same session.

A physical meaning of Formula 4 displays an overall average severity of the stagnant blood flow. In essence, a plurality of recordings are aggregated into a single virtual recording, with a percentage average value representing a stagnant blood flow level of the virtual recording, namely, the average severity of the stagnant blood flow across all recordings in this session.

The data processing module further includes fifth category data C, namely multi-recording percentage average value data C. C contains at least three sub-items, C1, C2, and C3.
C3: C3 is obtained by summing a completely normal percentage Q3 of all single recordings of the same measured object in the same session and dividing a sum by the total number of all recordings of the same measured object in the same session, referred to as a multi-recording normal blood flow percentage average value C3, as shown in Formula 5,

$$C 3 = \{ [ {\sum }_{r = 1}^{t} Q 3 \% ( r ) ] / t \} * 100 \% ,$$

where r=1,2,3 . . . t.; r represents a natural number sequence of recordings with Q3 present; for recordings without Q3, the value is 0; and t represents the total number of recordings for the same measured object in the same session.

A physical meaning of Formula 5 is to display an overall average recovery degree of the normal blood flow; or is actually to aggregate a plurality of recordings into a single virtual recording, with a percentage average value representing the recovery degree of the normal blood flow in this virtual recording, namely, an average recovery degree across all recordings in this session.

The data processing module further includes fifth category data C, namely multi-recording percentage average value data C, and C also contains a sub-item C4 in addition to C1, C2, and C3,
that is, Formula 6: C4=C1%:C2%:C3%.

Formula 6 reflects a mutual proportion C4 among the overall high-velocity blood flow percentage average value C1, the stagnant blood flow percentage average value C2, and the normal blood flow percentage average value C3 of all recordings acquired for the same measured object in the same session.

Embodiments of a device and method for a microcirculatory flow velocity quantitative indicator and a data detection and calculation system are provided. The system includes a microcirculatory observation instrument, a computer, or another display device, a pressure control and image stabilization apparatus, software, an acquisition module, a storage module, and a data processing module. The acquisition module observes and records blood flow in one or more pressure artifact-free sites with clearly visible vessels at the sublingual mucosa or other observable microcirculation sites of a subject in a resting state, with the aid of software or other display methods, using the microcirculatory observation instrument and the computer or another display device. The storage module stores recordings of one or more selected microcirculatory observation regions on a computer hard disk or other media using software or other means. The data processing module includes first category indicator A, which divides a microcirculatory blood flow velocity V in a single vessel into different ranges based on a velocity magnitude, to form the first category indicator A corresponding to each velocity range, which is also referred to as a velocity-related factor a for ease of understanding and calculation; and a is assigned different values based on different blood flow velocity ranges.

The data processing module includes: determining a value of a related proportional factor b of a second category indicator B for each single recording and a value of a diameter-related factor indicator d separately after a value of a for each recording is determined An indicator of the proportional factor b means a ratio of an area occupied by a vessel in a single recording within a different flow velocity range to a single recording area within an instrument's field of view at a single location, or a ratio of a length of a vessel within a different flow velocity range to a total vessel length, or a ratio of a density of a vessel within a different flow velocity range to a density of all vessels within a single recording area, where the density means a value obtained by dividing the total vessel length by the single recording area. The value of the diameter-related factor indicator d is assigned different numerical values vessels based on different vessel diameter ranges. A value of Qn % is calculated using Formula (1); values of C1 and C2 of the fifth category data C are calculated using Formulas (3) and (4); and a value of P is calculated using Formula (2).

The pressure control and image stabilization apparatus that is adjustable is installed on a microcirculatory microscope probe. The pressure control and image stabilization apparatus includes a positioning apparatus installed on a microcirculatory microscope elongated probe 1, the positioning apparatus includes a positioning apparatus front end 4 and a positioning apparatus rear end 6, and both of the positioning apparatus front end 4 and positioning apparatus rear end 6 are sleeves. Centers of the positioning apparatus front end and the positioning apparatus rear end are each provided with a through hole, allowing the microcirculatory microscope elongated probe or a disposable tapered protective apparatus for the elongated probe to pass through. The positioning apparatus rear end 6 is close to a microcirculatory instrument main unit 7. The positioning apparatus front end 4 and the positioning apparatus rear end 6 are fixed together or connected separately. The positioning apparatus can reciprocate along an axial direction 2 of the elongated probe of the microcirculatory instrument main unit and be fixed after being adjusted to a suitable position. The positioning apparatus front end 4 is provided with a flange 4-1 that serves as a bracket.

The pressure control and image stabilization apparatus is installed on a microcirculatory microscope probe to acquire stable, clear, and pressure-artifact-free microcirculation recordings from the subject at a resting state, which are stored on a computer hard disk or other media.

The length of the pressure control and image stabilization apparatus on the microcirculatory microscope is adjusted so that the microcirculatory microscope elongated probe just contacts a sublingual mucosa and is blocked by outer lips, allowing clear images to be seen without excessive pressure causing artificial stagnant blood flow artifacts.

A device and method for a microcirculatory flow velocity quantitative indicator and data detection calculation system are provided. A method for selecting different blood flow velocity ranges is: selecting a recording M with the fastest blood flow velocity among all clear, stable, pressure-artifact-free microcirculation recordings acquired for the same measured object in the same session; selecting a vessel M1 with the fastest blood flow velocity from the recording M as a measured target, where a vessel diameter is not considered for this fastest blood flow velocity, which is not a velocity of pulse blood flow, but rather a basic blood flow velocity as described in the present invention; and measuring a velocity after selecting the vessel with the fastest blood flow velocity, then determining whether this fastest blood flow velocity falls within a range of high-velocity blood flow Vd1, Vd2, Vd3, stagnant blood flow Vz4, Vz5, Vz6, or normal blood flow velocity Vc, and determining a type of infection accordingly.

Note: This fastest blood flow velocity is not the velocity of the pulse blood flow, but is the average velocity of baseline blood flow as previously described.

In an embodiment, when all blood flow velocities within a recording's field of view fall within the high-velocity blood flow range, the measured object is considered to suffer complete mild-to-moderate infection or interpreted as 100% mild-to-moderate infection; when all blood flow velocities within a recording's field of view fall within the stagnant blood flow range, the measured object is considered to suffer complete severe infection or interpreted as 100% severe infection; or when all blood flow velocities within a recording's field of view fall within the normal blood flow velocity range, the measured object is considered to be completely normal or interpreted as 100% normal.

Q1% is a high-velocity blood flow percentage, Q1%=(a1*b*d*g) %, Q2% is a stagnant blood flow percentage, Q2%=(a2*b*d*g) %, Q3% is a normal blood flow percentage, and Q3%=(a3*b*d*g) %.

All related factors a, b, d, g are specifically assigned based on the above principles and empirical formula-“Feng Xinghuai-Bernoulli-WarmShock” Principle.

### Embodiment 1

Ultra-early detection of sepsis (analysis of five recordings from the same patient in the same session and follow-up recordings):

Recording 1: High-velocity blood flow at 1,215 μm/s. Therefore, a flow velocity-related factor a of the first category indicator A is a=0.5, a high-velocity blood flow proportional factor is b=0.25, the high-velocity blood flow includes large, medium, and small vessels, and therefore the diameter-related factor is d=1, the standard factor is g=1. According to Formula 1: Qn %=(a*b*d*g) %, n=1,2,3 . . . natural number sequence; result: Q1%=(0.5*0.25*1*1)=0.125=12.5%, interpreted as: 12.5% high-velocity blood flow, or 12.5% mild-to-moderate infection.

Recording 2: A normal blood flow velocity, Q3%=(1*1**0)=0, a3=1, b=1, d=1, standard factor g=0, interpreted as: a normal blood flow velocity or 0% infection

Recording 3: Only one large vessel in each of quadrants 2 and 4 shows suspected high-velocity blood flow, with V=1,208 μm/s and V=1,280 μm/s respectively; two large vessels account for 30% of the total vessels, and therefore the flow velocity-related factor of the first category indicator A is a=0.5, the high-velocity blood flow proportional factor is b=0.3, high-velocity blood flow includes only large vessels, and therefore the diameter-related factor is d=0.3, the standard factor is g=1. According to Formula 1: Qn %=(a*b*d*g) %, n=1,2,3 . . . natural number sequence; result: Q1%=(0.5*0.3*0.3)=0.045=4.5%, interpreted as: 4.5% high-velocity blood flow, or 4.5% mild-to-moderate infection

Recording 4: Large, medium, and small vessels all exhibit suspected high-velocity blood flow, with V approximately equal to 1,000 μm/s (image is blurred and cannot be measured, and template comparison is used). The velocity-related factor of the first category indicator A is a=0.5, the high-velocity blood flow proportional factor is b=1, the diameter-related factor is d=1, and the standard factor is g=1. According to Formula 1, Q11%=(0.5*1*1*1*1)=0.5=50%, interpreted as: 50% high-velocity blood flow or 50% mild to moderate infection.

Recording 5: Two vessels exhibit suspected high-velocity blood flow. The velocity-related factor of the first category indicator A is a=0.5, the high-velocity blood flow proportional factor is b=50%, the diameter-related factor d is d=0.3, and the standard factor is g=1. According to Formula 1, Q1%=(0.5*0.5*0.3*1)=0.075=7.5%, interpreted as: 7.5% high-velocity blood flow or 7.5% mild to moderate infection.

According to Formula 3, the multi-recording high-velocity blood flow percentage average is a multi-recording mild to moderate infection average percentage C1.

C1=(0.125+0+0.045+0.5+0.075)/5=0.149, interpreted as 14.9% high-velocity blood flow or 14.9% mild to moderate infection.

Based on the above indicators, it can be learned that: 1. suspected high-velocity blood flow has already been detected, indicating an early stage of septic infection; 2. according to calculation of the multi-recording average percentage indicator and data C, it can be learned that a degree of high-velocity blood flow, namely, an overall infection level, is mild. 3. C1=14.9% indicates that the degree of high-velocity blood flow, namely, septic infection, is in an ultra-early or early stage.

In this case, clinical examination shows patient's procalcitonin (PCT)=1.1 ng/mL.

Analysis of recordings from the same patient 12 hours later:

Recording 1: High-velocity blood flow at 2,330 μm/s. Therefore, the velocity-related factor of the first category indicator A is a=1, the high-velocity blood flow proportional factor is b=0.5, the high-velocity blood flow includes large, medium, and small vessels, and therefore the diameter-related factor is d=1, and the standard factor is g=1. According to Formula 1: Qn %=(a*b*d*g) %, n=1,2,3 . . . natural number sequence; result: Q1%=(1*0.5*1*1)=0.5=50%, interpreted as: 50% high-velocity blood flow or 50% mild to moderate infection.

Recording 2: High-velocity blood flow at 1,747 μm/s. Therefore, the velocity-related factor of the first category indicator A is a=1, the high-velocity blood flow proportional factor is b=0.5, and high-velocity blood flow includes large, medium, and small vessels. Therefore, the diameter-related factor is d=1, and the standard factor is g=1. According to Formula 1: Qn %=(a*b*d*g) %, n=1,2,3 . . . natural number sequence; result: Q1%=(1*0.5*1*1)=0.5=50%, interpreted as: 50% high-velocity blood flow or 50% mild to moderate infection.

Recording 3: High-velocity blood flow at 1,305 μm/s. Therefore, the velocity-related factor of the first category indicator A is a=0.8, the high-velocity blood flow proportional factor is b=0.5, and the high-velocity blood flow includes large, medium, and small vessels. Therefore, the diameter-related factor is d=1, and the standard factor is g=1. According to Formula 1: Qn %=(a*b*d*g) %, n=1,2,3 . . . natural number sequence; result: Q1%=(0.8*0.5*1*1)=0.4=40%=40% high-velocity blood flow; or interpreted as: 40% mild to moderate infection.

According to Formula 3, the multi-recording high-velocity blood flow average percentage is a multi-recording mild to moderate infection average percentage C1.

C1=(0.5+0.5+0.4)/3=0.47=47% high-velocity blood flow, or interpreted as 47% mild to moderate infection.

Conclusion: The high-velocity blood flow average percentage of all three recordings, namely, the mild to moderate infection average percentage C1, is 47%, indicating moderate infection.

Compared with 14.9%, mild to moderate infection, 12 hours earlier, the condition has worsened.

In this case, clinical examination shows procalcitonin (PCT)=15.36 ng/mL.

### Embodiment 2

Installation and use of the pressure control and image stabilization apparatus (referred to as the positioning apparatus):

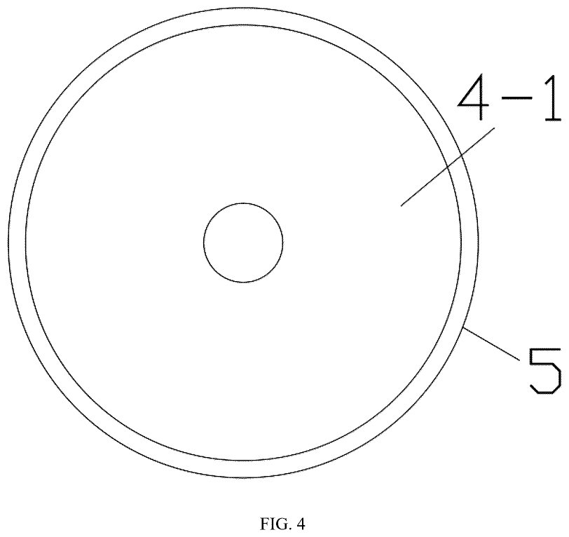

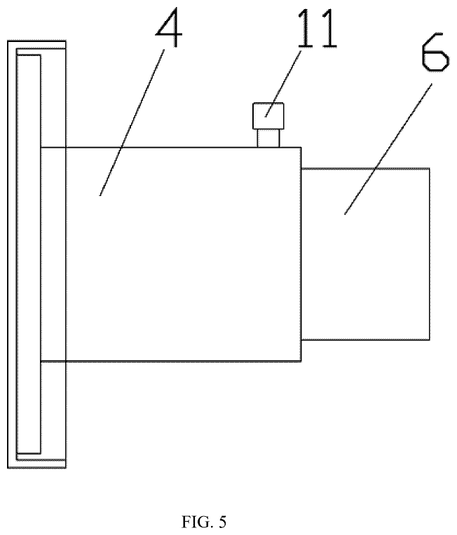

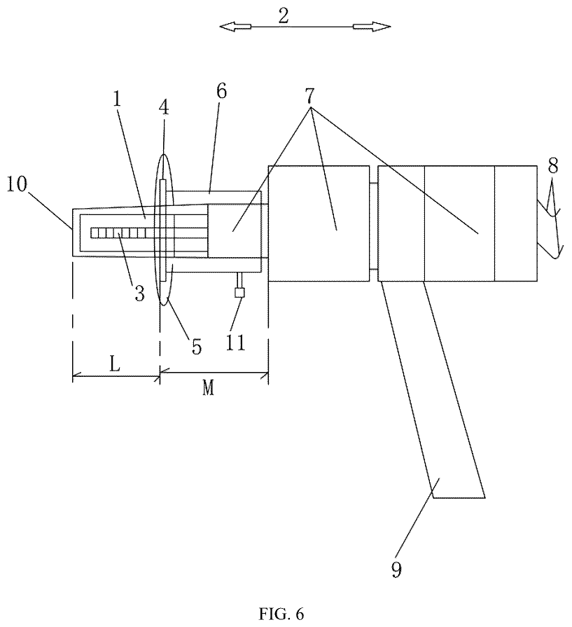

As shown in FIG. 3 to FIG. 6, an image stabilization and probe pressure control apparatus for a handheld microcirculatory instrument includes a positioning apparatus installed on a microcirculatory microscope elongated probe 1. The positioning apparatus is installed within a radius of 100 mm centered on an axis of the microcirculatory microscope elongated probe 1. The positioning apparatus includes a positioning apparatus front end 4 and a positioning apparatus rear end 6, both the positioning apparatus front end 4 and the positioning apparatus rear end 6 are sleeves. Centers of the positioning apparatus front end and positioning apparatus rear end are each provided with a through hole, allowing the microcirculatory microscope elongated probe or a disposable tapered protective apparatus for the elongated probe to pass through. The positioning apparatus rear end 6 is close to a microcirculatory instrument main unit 7. The positioning apparatus front end 4 and positioning apparatus rear end 6 are fixed together or are connected separately. Refer to FIG. 3, FIG. 4, and FIG. 5. The positioning apparatus front end 4 is provided with a flange 4-1 that acts as a bracket. The flange 4-1 can be fitted and pressed against a position near an observed target. For example, when sublingual microcirculation is observed, the flange 4-1 can be pressed against facial skin near an oral cavity to serve as a fulcrum, providing stability and reliability.

The positioning apparatus can move reciprocally along an axial direction 2 of the elongated probe of the microcirculatory instrument main unit and can be fixed after being adjusted to a proper position. Through such reciprocating adjustment, after a working distance L between the positioning apparatus front end 4 and a front end of a disposable tapered protective apparatus 10 for the elongated probe is adjusted, the positioning apparatus front end 4 and the front end of the disposable tapered protective apparatus 10 are simultaneously fixed to the microcirculatory instrument main unit 7.

### Embodiment 3

### Three Recordings, Patient Bed Number: 325110

First, observation of all three recordings reveals extensive stagnant blood flow, indicating severe infection. Formula 1 is used: Qn %=(a*b*d*g) %, to calculate a Q2% value.

Q2%=(1*1*1*100%*1)=1=100%
recording 2: a is set to 1, b is calculated as 100%, d=1, g=1,

Q2%=(1*1*1*100%*1)=1=100%
Recording 3: a is set to 1, b is calculated as 50%, d=1, g=1,

Q2%=(1*1*50%*1)=0.5=50%.

Next, Formula 4 is used to calculate a stagnant blood flow average percentage C2, namely, an average percentage C2 of multi-recording severe infection or non-infectious shock.

C2=(1+1+0.5)/3=0.83=83%,

Calculation shows a severe case proportion as high as 83%, but blood pressure still does not decrease significantly. The patient died two days later.

The above descriptions are only exemplary embodiments of the present invention and are not used to limit the present invention. Any modifications, equivalent substitutions, improvements, and the like made within the spirit and principle of the present invention should be included within the protection scope of the present invention.

1. A microcirculatory blood flow monitoring system, comprising: a microcirculatory observation instrument, an acquisition module, a storage module, and a data processing module, wherein the acquisition module observes blood flow and acquires blood flow data at one or more observable microcirculation sites on a subject using the microcirculatory observation instrument;
the storage module stores the blood flow data acquired by the acquisition module on a storage medium; the data processing module comprises first category data and a corresponding first-category indicator A, and divides the first category data, namely, a microcirculatory blood flow velocity V in a single vessel, into different ranges based on a velocity magnitude, to form the first-category indicator A corresponding to each velocity range, and the first-category indicator A is corresponding to a velocity-related factor a; the velocity-related factor a is assigned different values based on different blood flow velocity ranges; an assignment range for the velocity-related factor a is between greater than or equal to 0 and less than or equal to 1, the velocity-related factor a comprises one or more of the following related sub-factors: a high-velocity blood flow-related sub-factor a1, a stagnant blood flow-related sub-factor a2, and a normal blood flow-related sub-factor a3.

2. The microcirculatory blood flow monitoring system according to claim 1, wherein
the high-velocity blood flow-related sub-factor a1 is corresponding to high-velocity blood flow with a microcirculatory blood flow velocity V greater than 1,000 μm/s and is divided into three levels:
high-velocity Level 1 Vd3: a high blood flow velocity greater than or equal to 1,500 μm/s;
high-velocity Level 2 Vd2: a high blood flow velocity greater than or equal to 1,300 μm/s and less than 1,500 μm/s; and
high-velocity Level 3 Vd1: a high blood flow velocity greater than 1,000 μm/s and less than 1,300 μm/s;
the stagnant blood flow-related sub-factor a2 is corresponding to stagnant blood flow with the microcirculatory blood flow velocity V less than or equal to 90 μm/s, which is divided into three levels:
stagnant Level 1 Vz4: a stagnant blood flow velocity less than or equal to 30 μm/s;
stagnant Level 2 Vz5: a stagnant blood flow velocity greater than 30 μm/s and less than or equal to 60 μm/s; and
stagnant level Vz6: stagnant blood flow velocity greater than 60 μm/s and less than or equal to 90 μm/s;
the normal blood flow-related sub-factor a3 is corresponding to normal blood flow with the microcirculatory blood flow velocity V less than or equal to 1,000 μm/s and greater than 90 μm/s; and
based on the above different levels of velocities, corresponding sub-sub factors are assigned to the velocity-related sub-factors: the velocity-related sub-factor a1 is divided into sub-sub factors a1-1, a1-2, and a1-3; and the velocity-related sub-factor a2 is divided into sub-sub factors a2-1, a2-2, and a2-3.

3. The microcirculatory blood flow monitoring system according to claim 1, wherein
the data processing module further comprises second category data and a corresponding second category indicator B; the second category indicator B is corresponding to a proportional factor b, an indicator of the proportional factor b means a ratio of an area occupied by a vessel in a single recording within a different flow velocity range to a single recording area within an instrument's field of view at a single location, or a ratio of a length of a vessel within a different flow velocity range to a total vessel length, or a ratio of a density of a vessel within a different flow velocity range to a density of all vessels within a single recording area, wherein the density means a value obtained by dividing the total vessel length by the single recording area; and the proportional factor b serves as both the second category data and the indicator; and a value range for the proportional factor b is between greater than or equal to 0 and less than or equal to 1.

4. The microcirculatory blood flow monitoring system according to claim 1, wherein
the data processing module further comprises third category data and a corresponding third category indicator D; the third category data comprises vessel diameter range classification, and the third category indicator D comprises a diameter-related factor d corresponding to a vessel diameter range, wherein vessel data within a different diameter range is corresponding to a respective diameter-related factor d; the diameter-related factor d is assigned different values based on different vessel diameter ranges; and a value range for the diameter-related factor d is between greater than or equal to 0 and less than or equal to 1.

5. The microcirculatory blood flow monitoring system according to claim 1, wherein the data processing module further comprises a standard factor indicator g for high-velocity blood flow, stagnant blood flow, or normal-velocity blood flow, the standard factor indicator is g=1 when all blood flow velocities fall within a high-velocity blood flow range or all fall within a stagnant blood flow range; or the standard factor indicator is g=0 when all blood flow velocities fall within a normal-velocity blood flow range.

6. The microcirculatory blood flow monitoring system according to claim 1, wherein
the data processing module further comprises fourth category data, namely, calculation data Qn % of related factors:
Qn % is calculated using any one of the following Formula 1-1, Formula 1-2, or Formula 1-3:
Qn %=(a*b) % Formula 1-1:
Qn %=(a*b*d) % Formula 1-2:
Qn %=(a*b*d*g) % Formula 1-3:
wherein n=1,2,3 . . . , which is a natural number sequence, when n=1, Q1% is a percentage of the high-velocity blood flow; when n=2, Q2% is a percentage of the stagnant blood flow; when n=3, Q3% is a percentage of the normal blood flow; a is the velocity-related factor, b is the proportional factor, d is the diameter-related factor, and g is the standard factor indicator.

7. The microcirculatory blood flow monitoring system according to claim 1, wherein
a value range of the flow-related velocity a is 0-1: as a blood flow velocity is approaching 1,500 μm/s from a lower value, a high-velocity blood flow-related sub-factor a1 is approaching 1; as the blood flow velocity is greater than 1,500 μm/s, a1 is assigned a value of 1; and as the blood flow velocity is approaching 0-30 μm/s from above 30 μm/s, the stagnant blood flow-related sub-factor a2 is approaching 1; within a 0-30 μm/s (comprising 30 μm/s) velocity range, a2=1, the normal blood flow-related sub-factor is a3=1;
a value range of the diameter-related factor d is 0-1: microcirculatory vessels with a diameter less than or equal to 20 m are classified as small vessels, microcirculatory vessels with a diameter greater than 20 m and less than or equal to 60 m are classified as medium vessels, and microcirculatory vessels with a diameter greater than 60 m are classified as large vessels; and when high-velocity blood flow occurs in large, medium, and small vessels, a sub-factor related to d is d1=1, which is assigned within 0-1 in other cases; or when stagnant blood flow occurs in the large, medium, and small vessels, the sub-factor related to d is d2=1, which is assigned within 0-1 in other cases.

8. The microcirculatory blood flow monitoring system according to claim 1, wherein
the data processing module further comprises fifth-category data C, namely, multi-recording percentage average value data C; and C comprises at least one sub-item C1; and
C1 is obtained by summing a single recording high-velocity blood flow percentage Q1 for all recordings of the same measured object in the same session and dividing a sum by the total number of recordings for the subject in the session, and C1 is referred to as a multi-recording high-velocity blood flow percentage average value C1, as shown in Formula 3,
$$C 1 = \{ [ {\sum }_{i = 1}^{t} Q 1 \% ( i ) ] / t \} * 100 \% ,$$
i=1,2,3 . . . t.; i represents a natural number sequence of recordings with Q1% present; for recordings without Q1%, a value is 0; and t represents the total number of recordings for the same measured object in the same session.

9. The microcirculatory blood flow monitoring system according to claim 8, wherein
the data processing module further comprises fifth category data C, namely multi-recording percentage average value data C, and C comprises at least two sub-items, C1 and C2;
C2: C2 is obtained by summing a stagnant blood flow percentage Q2% of each single recording stagnant blood flow indicator of each recording for the same measured object in the same session and by dividing a sum by the total number of all recordings of the same measured object in the same session, referred to as the multi-recording stagnant blood flow percentage average value C2, as shown in Formula 4,
$$C 2 = \{ [ {\sum }_{w = 1}^{t} Q 2 \% ( w ) ] / t \} * 100 \% ,$$
w=1,2,3 . . . t.; w represents a natural number sequence of recordings with Q2% present; or recordings without Q2%, a value is 0; and t represents the total number of recordings for the same measured object in the same session.

10. A microcirculatory blood flow monitoring device, wherein the system comprises a microcirculatory observation instrument, a computer, a pressure control and image stabilization apparatus, an acquisition module, a storage module, and a data processing module; the acquisition module observes blood flow and acquires blood flow data at one or more observable microcirculation sites on a subject using the microcirculatory observation instrument; the storage module stores the blood flow data acquired by the acquisition module on a storage medium; the data processing module comprises first category data and a corresponding first-category indicator A, and divides a microcirculatory blood flow velocity V in a single vessel into different ranges based on a velocity magnitude, to form the first-category indicator A corresponding to each velocity range, and the first-category indicator A is corresponding to a velocity-related factor a; and an assignment interval for a is between greater than or equal to 0 and less than or equal to 1.

11. The microcirculatory blood flow monitoring device according to claim 10, wherein the data processing module comprises: determining a value of a related proportional factor b of a second category indicator B for each single recording and a value of a diameter-related factor d thereof separately after a value of a for each recording is determined; an indicator of the proportional factor b means a ratio of an area occupied by a vessel in a single recording within a different flow velocity range to a single recording area within an instrument's field of view at a single location, or a ratio of a length of a vessel within a different flow velocity range to a total vessel length, or a ratio of a density of a vessel within a different flow velocity range to a density of all vessels within a single recording area, wherein the density means a value obtained by dividing a part of a length or a total length of a vessel by the single recording area; an assignment interval for b is between greater than or equal to 0 and less than or equal to 1; d is assigned different values based on different vessel diameter ranges, and an assignment interval for d is between greater than or equal to 0 and less than or equal to 1; and a value of Qn % is calculated using Formula 1; and then fifth category data C, C1 and C2, is calculated based on Formula 3 and Formula 4.

12. The microcirculatory blood flow monitoring device according to claim 10, wherein the microcirculatory observation instrument is a microcirculatory microscope probe, the pressure control and image stabilization apparatus that is adjustable is installed on a microcirculatory microscope probe, the pressure control and image stabilization apparatus comprises a positioning apparatus installed on a microcirculatory microscope elongated probe, the positioning apparatus comprises a positioning apparatus front end and a positioning apparatus rear end, both of the positioning apparatus front end and the positioning apparatus rear end are sleeves, centers of the positioning apparatus front end and the positioning apparatus rear end are each provided with a through hole, allowing the microcirculatory microscope elongated probe or a disposable tapered protective apparatus for the elongated probe to pass through, and the positioning apparatus rear end is close to a microcirculatory instrument main unit; the positioning apparatus front end and the positioning apparatus rear end are fixed together or connected separately; the positioning apparatus is capable of reciprocating along an axial direction of the elongated probe of the microcirculatory instrument main unit and is fixed after being adjusted to a suitable position, and the positioning apparatus front end is provided with a flange that serves as a bracket.
